(19) United States
(12) Patent Application Publication
(10) Pub. No.: US 2022/0404374 A1
(43) Pub. Date: Dec. 22, 2022

(54) METHOD FOR DETERMINING WHETHER OR NOT A SUBJECT HAS A PARKINSONIAN CONDITION

(71) Applicant: UNIVERSITE DU LUXEMBOURG, ESCH-SUR-ALZETTE (LU)

(72) Inventors: Jean-Pierre Trezzi, Rodange (LU); Paul Wilmes, Bettembourg (LU)

(21) Appl. No.: 17/776,010

(22) PCT Filed: Nov. 12, 2020
(86) PCT No.: PCT/EP2020/081832

§ 371 Date: May 11, 2022

(30) Foreign Application Priority Data
Nov. 12, 2019 (LU) LU101476

(51) Int. Cl. G01N 33/68

(52) U.S. Cl. CPC G01N 33/6896; G01N 2560/00; G01N 2800/2835; G01N 2800/52

(57) ABSTRACT

The present invention relates to a method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control. The present invention further also relates to a method for monitoring the progression of a parkinsonian condition in a subject diagnosed with the parkinsonian condition and a method for assessing the efficacy of treatment of a parkinsonian condition in a subject diagnosed with the parkinsonian condition as well as a kit-of-parts for determining whether or not a subject has a parkinsonian condition.

The work leading to this invention received support from the Luxembourg Fonds National de la Recherche (FNR) under grant No. CORE 11333923 and the Michael J. Fox Foundation under grant No. 14701.

### TECHNICAL FIELD

The present invention relates to a method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control. The present invention further also relates to a method for monitoring the progression of a parkinsonian condition in a subject diagnosed with the parkinsonian condition and a method for assessing the efficacy of treatment of a parkinsonian condition in a subject diagnosed with the parkinsonian condition as well as a kit-of-parts for determining whether or not a subject has a parkinsonian condition.

### BACKGROUND OF THE INVENTION

Neurological disorders have become the leading cause of disability in the world, and Parkinson's disease (PD) is one of the most significant medical and social burdens of our time. With the aging population, the number of people suffering from PD is expected to double from 6.9 million in 2015 to 14.2 million in 2040 (Dorsey et al., 2018). PD is a complex, progressive, and widely systemic neurodegenerative disease characterized by a number of motor and non-motor symptoms. PD has a long (up to 20-30 years of) prodromal period, during which several non-motor features can develop, including impairment in olfaction, constipation and rapid eye movement sleep behaviour disorder (RBD) (Chaudhuri et al., 2009; Schrag et al., 2015).

Previous investigations of biomarkers in biological fluids (blood and cerebrospinal fluid) have neither yielded diagnostic nor progression markers for early detection (Simonsen et al., 2016; Mollenhauer Zimmermann et al., 2016). The underlying pathogenesis of PD is still elusive, and biomarkers are important for upcoming clinical trials with putative disease-modifying agents (Olanow et al., 2010).

The defining pathology in PD is the death of dopaminergic neurons in a part of the midbrain called the substantia nigra. Another hallmark of PD is an accumulation of neuronal inclusions, known as Lewy bodies, in various parts of the brain and body (such as the substantia nigra, cerebral cortex, dorsal nucleus of the vagus nerve, sympathetic ganglia, and the myenteric plexus of the intestines). These Lewy bodies contain misfolded alpha-synuclein, ubiquitin, complement proteins, and cytoplasmic structural proteins. The propagation of α-synuclein aggregations in the form of intracellular Lewy body inclusions in PD has been shown to start peripherally in the enteric nervous system (ENS; Braak et al., 2006) and the olfactory bulb (Braak et al., 2006; Braak et al., 2003) before affecting the central nervous system (CNS) resulting in a staged topographic ascending distribution pattern of intracerebral lesions.

In this context, emerging studies on the subject of PD and the microbiota have begun to focus on the potential mechanisms by which the microbiota contribute to the formation of alpha-synuclein pathogenic species in the ENS and CNS. Within the gut microbiome, PD-specific signatures have recently been reported (Heintz-Buschart et al., 2018; Scheperjans et al., 2015a; Scheperjans et al., 2015b). However, at present, it remains unclear whether the differences in the gut microbiome of PD patients are a consequence of the disease or causally related to the presence of causative microbial agents, such as toxin-producing organisms.

Despite the immediate research interest and the relatively easy accessibility (once standard operating procedures are established), not a single published study on the gut microbiome exists in the context of early and longitudinally followed PD, and at-risk subjects (RBD). The only previous studies of the gut microbiome of PD patients have used a phylogenetic marker sequencing approach (16S rRNA gene amplicon sequencing), which allows for the taxonomic identification and relative quantification of microorganisms, thereby not allowing a realistic assessment of the vitality, functional potential or functional activity of these taxa and therefore not conveying important disease-relevant information (Scheperjans et al., 2015b; Keshavarzian et al., 2015).

Independently of the microbiome, recent studies (Horvath et al., 2012) have shown that the ring-fused 2-pyridone molecule (FN075) inhibits fibrillation of amyloidogenic curli protein CsgA and interestingly stimulates alpha-synuclein amyloid fiber formation. Thereby, the Curlicide/Pilicide with a reactive ring-fused 2-pyridone seems to have an impact on alpha-synuclein aggregation/fibrillation, as observed in Parkinson's disease. Interestingly, in vivo approaches (Chermenina et al., 2015; Cairns et al., 2018) that delivered 2-pyridone fibrillization modulators to both mice and non-human primate were developed and resulted in symptoms mimicking early stages of Parkinson's disease. However, no correlation between the ring-fused 2-pyridone and the microbiome has been shown.

Furthermore, HMDB, the Human Metabolome Database, shows no entries for the central core moiety of the FN075, 2-pyridone or its tautomer 2-HP, in faecal samples.

The molecule 2-HP is a known co-factor of archaeal iron-containing hydrogenases (Vogt et al., 2008) and a degradation product of the abundant pesticide chlorpyrifos. Harishankar et al. (2013) shows that the chlorpyrifos is the main source of the metabolite 3,5,6-trichloro-2-pyridinol (TCP), which—in turn—may be transformed into 2-HP probably via dechlorination by bacteria, including members of the gut microbiome, such as Lactobacillus spp.

There is a need for new diagnostic and reliable methods, which enable to distinguish between PD and non-PD patients, which is qualitative, easy to access, reproductive and has low costs. The present invention addresses all these needs. Accordingly, the technical problem underlying the present application is to comply with these needs.

### SUMMARY OF THE INVENTION

The inventors of the present invention found 2-hydroxypyridine (2-HP or 2HP in the following) as a microbiome-derived multi-omic biomarker for Parkinson's disease diagnosis and progression and as a key molecule involved in the pathogenesis and progression of idiopathic PD. 2-Hydroxypyridine can be a faecal sample-derived biomarker for Parkinson's disease diagnosis and progression. The biomarker can be used alone or in combination, with a high predictive and prognostic value in various parkinsonian conditions selected from the group consisting of Parkinson's disease, Parkinsonism, Parkinson-plus syndrome, early-stage idiopathic Parkinson's disease (PD) and prodromal Rapid eye movement (REM) sleep behaviour disorder (RBD). This reveals 2-HP, an archaeal cofactor and pesticide degradation product, not only as being a key molecule involved in the pathogenesis and progression of idiopathic PD, but provides also a mechanistic insight of the involvement of 2-HP as a driver of Parkinson's disease pathology.

The present invention relates to a method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control. In a preferred embodiment of this method, the sample is a biological sample. In an even more preferred embodiment, the biological sample is a faecal sample. It is also preferred for the method for determining whether or not a subject has a parkinsonian condition, that the concentration of 2-hydroxypyridine is determined by mass spectrometry or NMR. In a further preferred embodiment of this method, the control is determined by measuring the concentration of 2-hydroxypyridine in samples obtained from at least 45, preferably at least 50 and more preferably at least 100, control subjects, wherein the control subjects are healthy control subjects characterized by not having the parkinsonian condition. It is also possible, that the method further comprises determining the concentration of beta-glutamic acid, conducting a neurological test or classification according to Unified Parkinson's Disease Rating Scale (UPDRS). It may also be preferred that the subject is a human.

The present invention also relates to a method for monitoring the progression of a parkinsonian condition in a subject diagnosed with the parkinsonian condition, comprising (i) determining the concentration of 2-hydroxypyridine in a sample obtained from the subject, wherein the determined concentration of 2-hydroxypyridine is compared to a control sample of the same subject. It is preferred for said method that the control sample has been obtained from the subject a certain time before step (i), more preferably wherein certain time means at least 6 months, even more preferably at least 1 year, before step (i). In a preferred embodiment of said method for monitoring the progression of a parkinsonian condition, the progression of the parkinsonian condition is positive, if the concentration of 2-hydroxypyridine has decreased compared to the control sample of the same subject or has maintained the same compared to the control sample of the same subject. It is also preferred for said method for monitoring the progression of a parkinsonian condition that the progression of the parkinsonian condition is negative, if the concentration of 2-hydroxypyridine has increased compared to the control sample of the same subject, more preferably wherein the concentration of 2-hydroxypyridine has significantly increased compared to the control sample of the same subject.

The present invention also relates to a method for assessing the efficacy of treatment of a parkinsonian condition in a subject diagnosed with the parkinsonian condition, comprising (i) determining the concentration of 2-hydroxypyridine in a sample obtained from the subject, wherein the determined concentration of 2-hydroxypyridine is compared to a control sample of the same subject. It is preferred for said method that the control sample has been obtained from the subject a certain time before step (i), more preferably wherein certain time means at least 6 months, even more preferably at least 1 year, before step (i). In a preferred embodiment of said method for assessing the efficacy of treatment of a parkinsonian condition, the efficacy of treatment of the parkinsonian condition is assessed as being positive, if the concentration of 2-hydroxypyridine has decreased compared to the control sample of the same subject or has maintained the same compared to the control sample of the same subject. It is also preferred for said method for assessing the efficacy of treatment of a parkinsonian condition, that the efficacy of treatment of the parkinsonian condition is assessed as being negative, if the concentration of 2-hydroxypyridine has increased compared to the control sample of the same subject, more preferably wherein the concentration of 2-hydroxypyridine has significantly increased compared to the control sample of the same subject.

The present invention also relates to a kit-of-parts for determining whether or not a subject has a parkinsonian condition, comprising means for determining the concentration of 2-hydroxypyridine in a sample obtained from the subject and a test system that is capable of determining whether or not said subject has a parkinsonian condition, based on the result of the determined concentration of 2-hydroxypyridine of said subject compared to a control.

### BRIEF DESCRIPTION OF THE DRAWINGS

FIG. 1 shows significant changes (p=0.0007, FDR=0.006) in the abundance of 2-HP. The levels of this metabolite are significantly higher in PD and RBD compared to healthy controls, but not significantly altered between PD and RBD. Semi-quantitative levels of 2-HP have been determined by GC-MS analysis from faecal samples derived from 45 PD patients, 30 iRBD subjects as well as 50 healthy control subjects.

FIG. 2 shows significant changes (p=0.00005, FDR=0.005) in the abundance of beta-glutamic acid. The levels of this metabolite are significantly higher in RBD compared to healthy controls, but not significantly altered between PD and controls. Semi-quantitative levels of beta-glutamic acid have been determined by GC-MS analysis from faecal samples derived from 45 PD patients, 30 iRBD subjects as well as 50 healthy control subjects.

FIG. 3 shows toxicity assays of 2-HP in HiTox strains of yeast expressing human alpha-synuclein. 2-HP is selectively toxic at doses of 1 mM to transgenic alpha-synuclein expressing yeast. FIG. 3A shows that in presence of raffinose, alpha-synuclein is not induced. A toxic dose-response can be observed. FIG. 3B shows that in presence of galactose, alpha-synuclein is induced, leading to growth defects. A toxic dose-response can be observed in the HiTox strains, but not in the control strain. FIG. 3C shows the growth curve in presence and absence of 2-HR. At higher concentration of 2-HP, the lag time in the control is prolonged. However, the yield is unaffected.

FIG. 4 shows quantitative alpha-synuclein aggregation assays in HiTox strains of yeast expressing human alpha-synuclein after treatment with 2-HP. The aggregation of alpha-synuclein is the key molecular hallmark of PD.

FIG. 5 shows the toxicity of 2-HP in enteric neurons. LD₅₀ seems to be located around 3 mM. In addition to the yeast model, the acute toxicity and alpha-synuclein induced aggregation of 2-HP in iPS cells differentiated to a proxy of the cells present in the enteric nervous system have been assessed. At 3 mM, the inventors have observed significant changes in the toxicity of 2-HP on these cells.

FIG. 6 shows semi-quantitative alpha-synuclein aggregation assays in enteric neurons after treatment with 2-HP. The following abbreviations have the meaning given in brackets: α-Syn (alpha-synuclein), α-Syn-F (fibrillary alpha-synuclein, i.e. alpha-synuclein aggregations), Hoechst (DNA staining), TUJ1 (general neuronal marker), TH (tyrosine hydroxylase), CC3 (cleaved caspase 3 staining, i.e. cell death marker, showing toxicity).

FIG. 7 shows the effects of 2-hydroxypyridine on IL-1b mRNA expression in murine BM DM model.

FIG. 8 shows the relative abundance of genus Methanobrevibacter in two cohorts.

FIG. 9 shows the concentrations of 2-hydroxypyridine in cell pellets from archaeal cultures (targeted GC/MS).

FIG. 10 shows an adhesive removal test in mice, 15 days post 2-HP injection. The following abbreviations have the meaning given in brackets: WT (wildtype mice), TG (transgenic mice overexpressing human alpha-synuclein). n=5 for each group/concentration.

### DETAILED DESCRIPTION OF THE INVENTION

The present invention provides a microbiome-derived multi-omic biomarker, 2-hydroxypyridine (2-HP or 2HP), relating to PD. In the present invention, the biomarker may be used for diagnostic purposes (early-stage PD and prodromal RBD). However, it may also be used to monitor PD progression and to assess response to treatment.

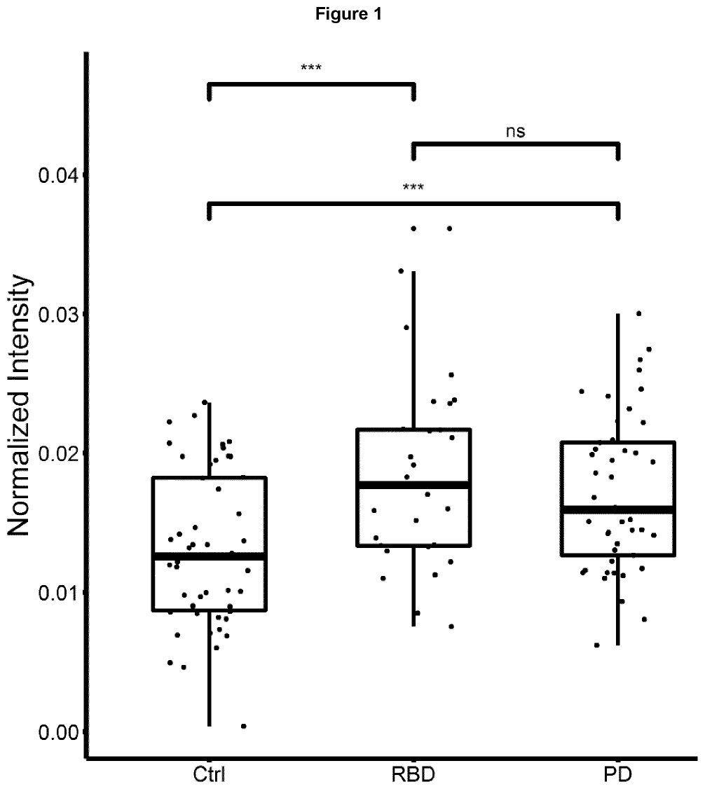

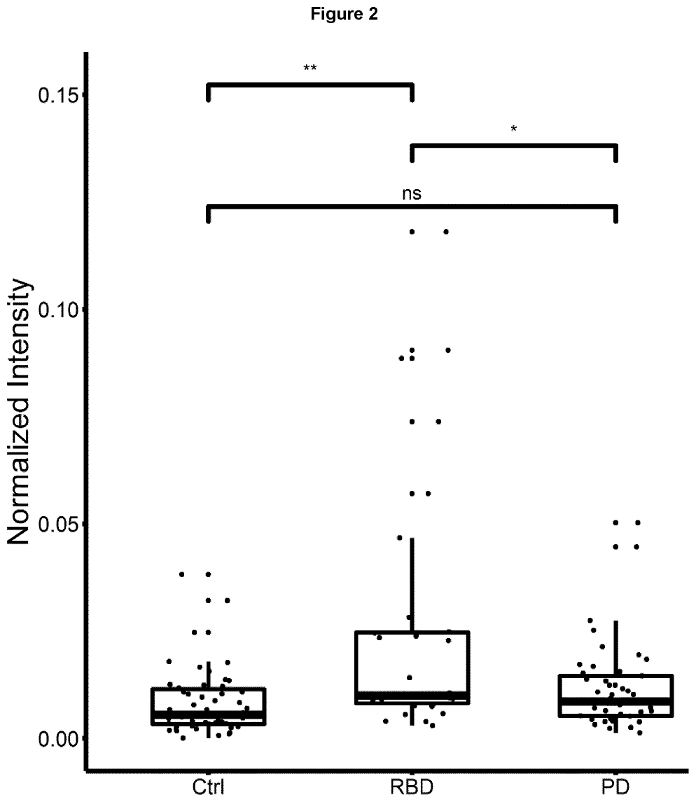

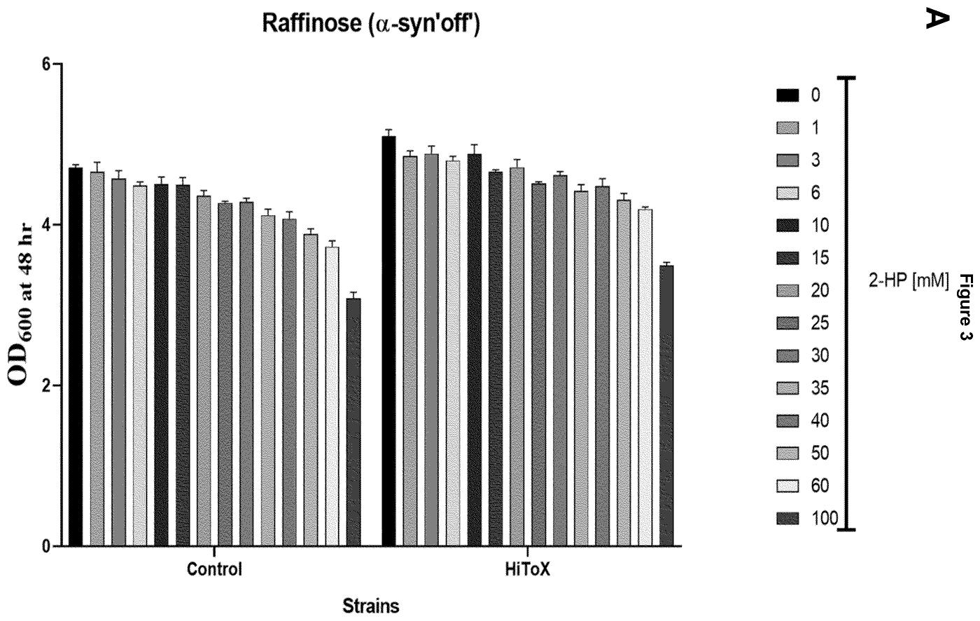

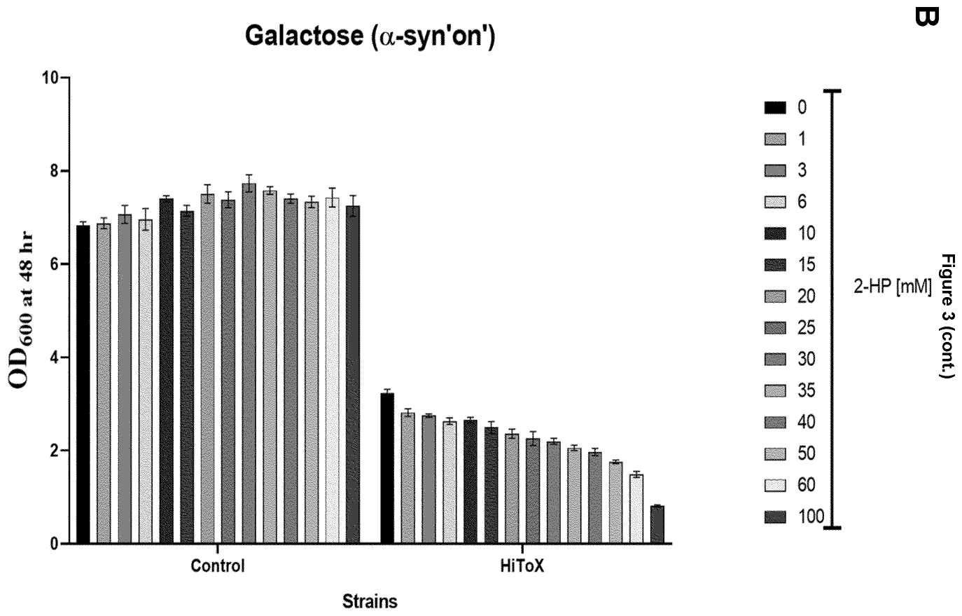

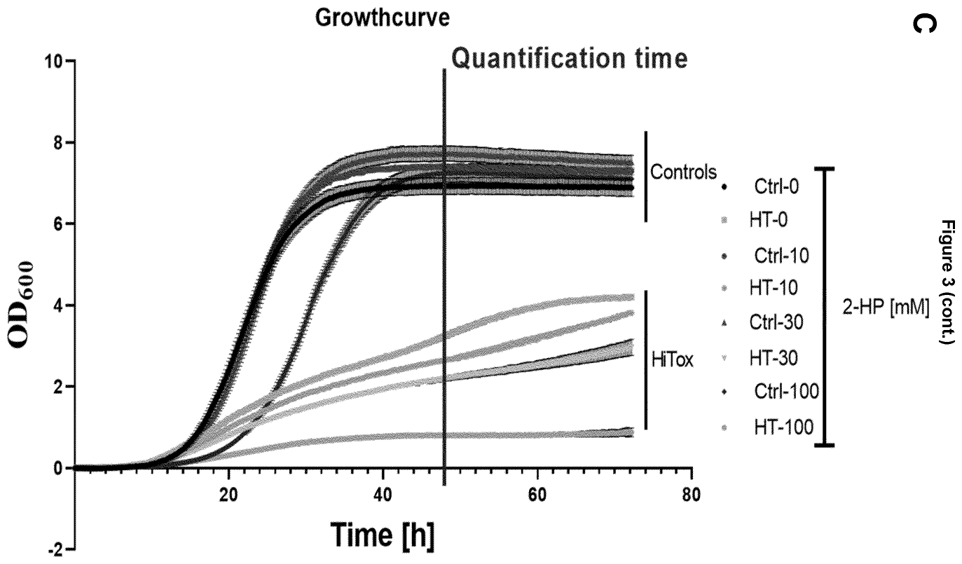

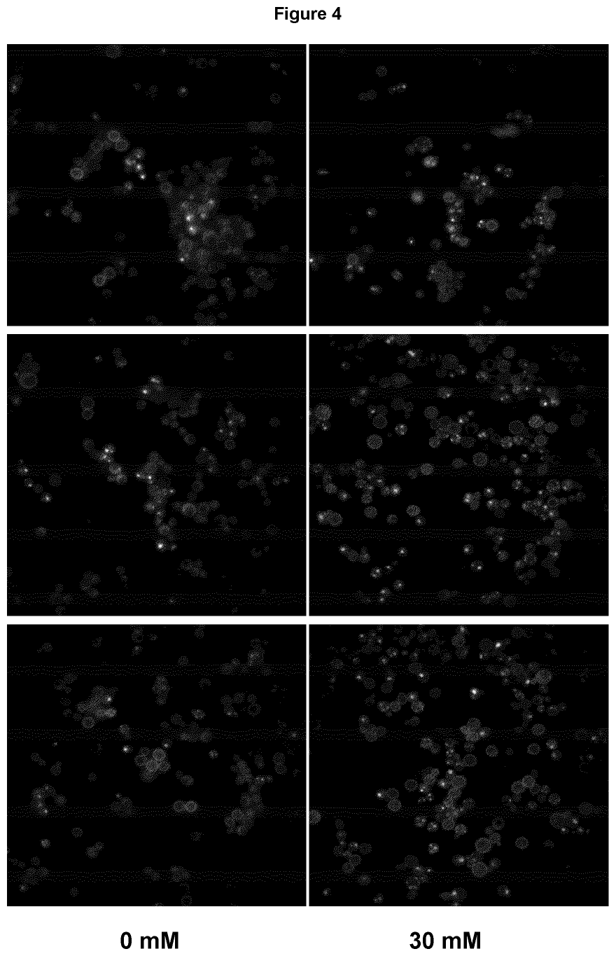

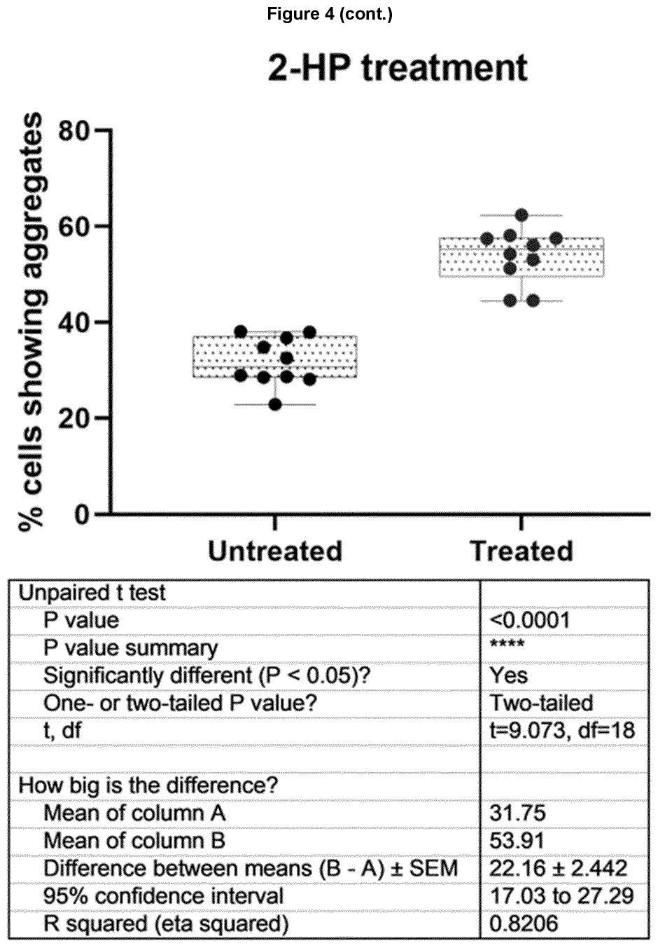

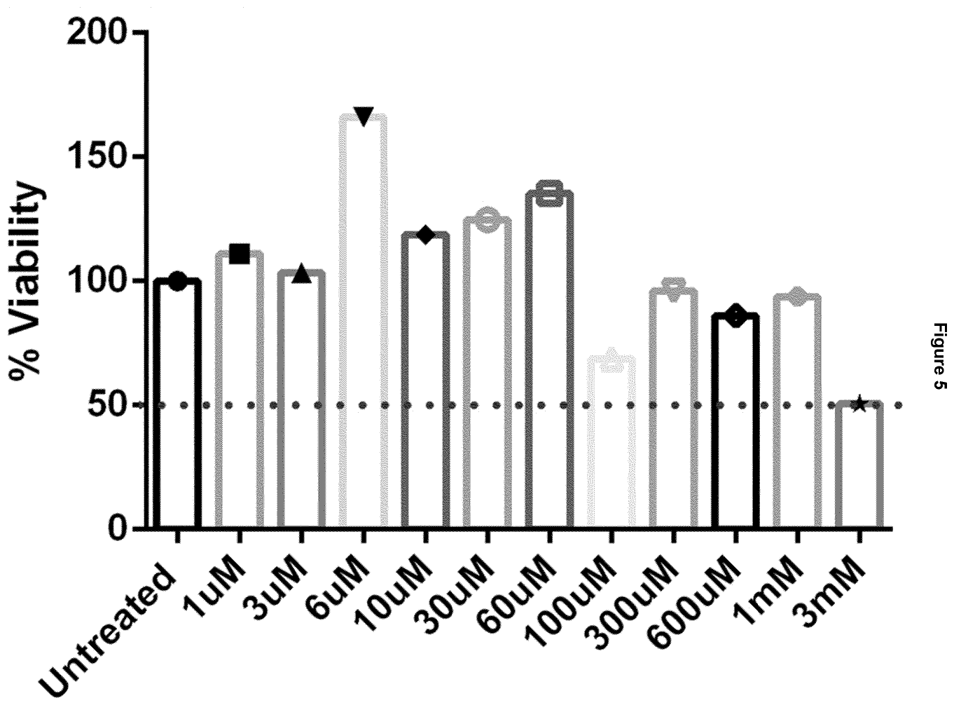

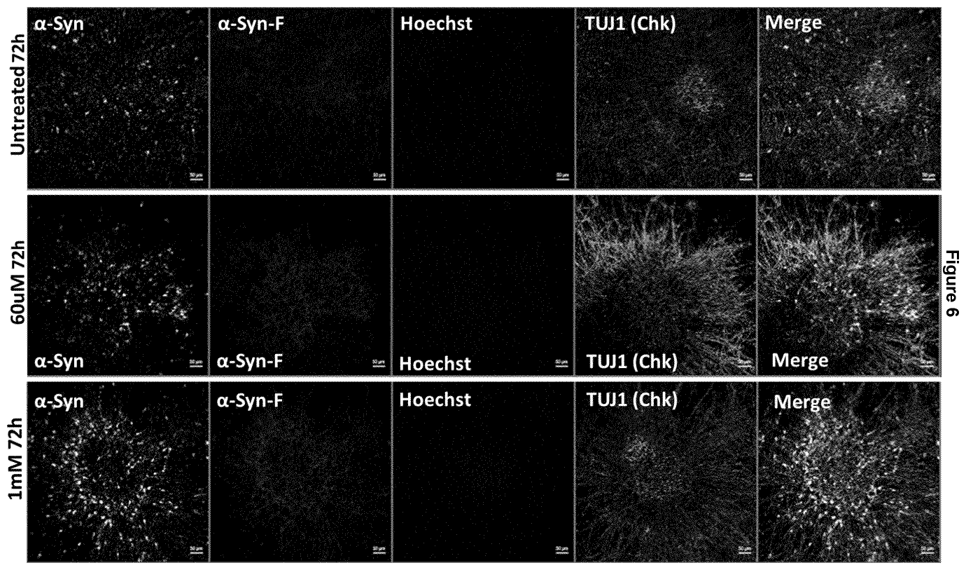

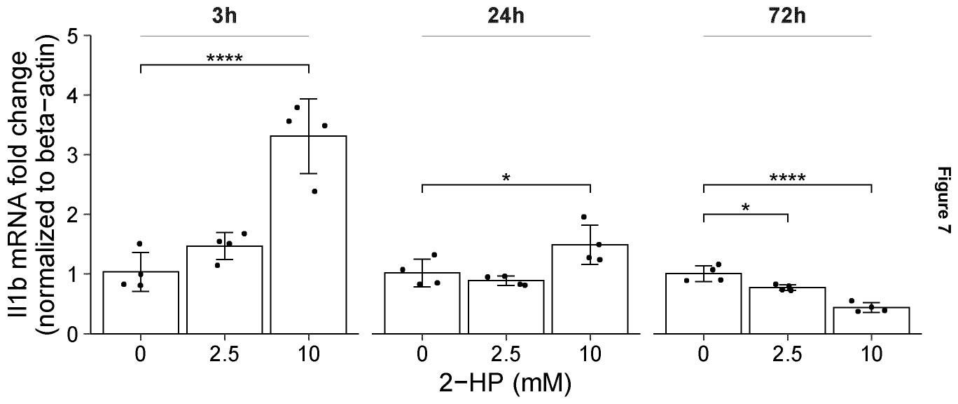

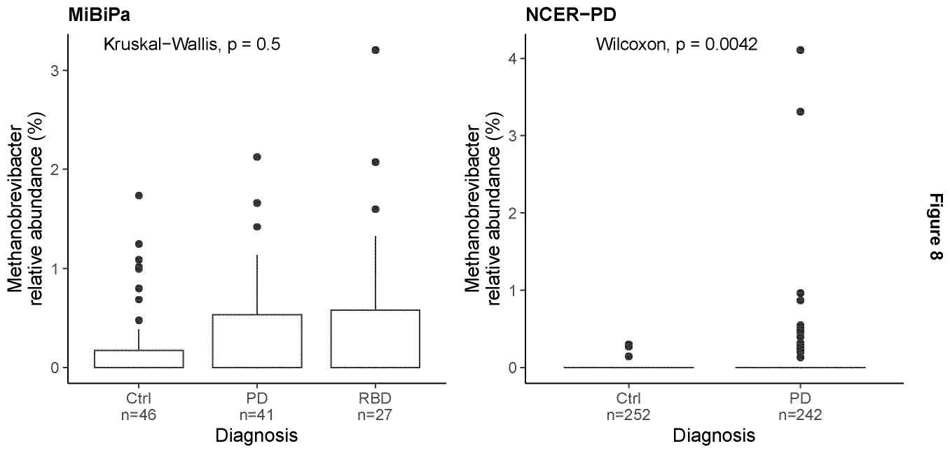

The gut microbiome and exposure to pesticides have been implicated in the pathogenesis of Parkinson's disease (PD). The inventors of the present invention performed an integrated meta-omic analysis of flash-frozen stool samples collected from idiopathic PD patients, idiopathic rapid eye movement sleep behavior disorder patients (RBD; a prodrome of PD), and healthy controls. The metabolomic data revealed elevated levels of two previously uncharacterized metabolites in PD and RBD patients: beta-glutamic acid (p=0.00005, FDR=0.005) and 2-hydroxypyridine (p=0.0007, FDR=0.006) (see FIGS. 1 and 2). The metabolite 2-hydroxypyridine (2-HP or 2HP, as abbreviated herein) is a known co-factor of archaeal iron-containing hydrogenases and a degradation product of the abundant pesticide chlorpyrifos. It is implicated in aggregation of alpha-synuclein, the key molecular hallmark of PD (see FIGS. 4 and 6). The metabolite is selectively toxic to transgenic alpha-synuclein expressing yeast and to enteric dopaminergic neurons (see FIGS. 3 and 5). It binds and inhibits mitochondrial enzymes involved in dopamine metabolism (in silico data). The results of the inventors implicate that 2-HP is a key molecule involved in the pathogenesis and progression of idiopathic PD. Moreover, there is evidence therefore that the majority of 2-HP may come from archaea (see Example 5). There is a strong correlation of the level of archaea Methanobrevibacter spp. with levels of 2-HP in PD as well as RBD patients (r=0.66) (see Example 6, FIG. 8). In parallel, the putative metabolite beta-glutamic acid is a known osmolyte molecule used in archaea, thus strongly supporting the evidence that archaea are involved in the pathogenesis and progression of idiopathic PD. For example, the inventors of the present invention have found a correlation of Methanobrevibacter spp. with beta-glutamic acid to be r=0.85 for the RBD and to be r=0.41 for PD.

The following detailed description refers to the accompanying Examples and Figures that show, by way of illustration, specific details and embodiments, in which the invention may be practised. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

The present invention relates to a method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control.

As used herein, in the context of the present invention, the term “determining whether or not” means that it is evaluated, if the condition under investigation or under examination is present or if that condition is not present with regard to a subject.

In the context of the present invention, the term “subject” means a human or an animal, wherein the animal may be an ape, a dog, a cat, a cow, a pig, a horse, a camel, a dromedary, a mouse, a rat, a rabbit, a sheep or a goat. In a preferred embodiment of the method according to the present invention, the subject is a human. In a more preferred embodiment of the present invention, the subject may be a human or patient, who has already been diagnosed with a parkinsonian condition or a human or patient that is suspected of having, or at risk of having or at risk of developing a parkinsonian condition.

In the context of the present invention, the term “determining a/the concentration” means to evaluate, assess or identify with any methods or means known to the person skilled in the art the amount or level of the compound under investigation, here 2-hydroxypyridine, in a specific sample.

As used herein, the term “parkinsonian condition” refers to a clinical/pathological condition (e.g., clinical situation), disease, state (e.g., pathological state) or syndrome comprising the following symptoms: tremor, bradykinesia, rigidity, and postural instability. Preferably, said parkinsonian condition is selected from the group consisting of: Parkinson's disease, Parkinsonism, Rapid Eye Movement Sleep Behavior Disorder (RBD) and Parkinson-plus syndrome.

As used herein, the term “parkinsonism” refers to a clinical/pathological condition (e.g., clinical situation) characterized by (e.g., consisting of) the following symptoms: Tremor, bradykinesia, rigidity and postural instability. Preferably, said Parkinsonism comprises one or more of the following conditions: Drug induced parkinsonism, toxin induced parkinsonism and secondary parkinsonism. Preferably, said drug induced parkinsonism is selected from the group consisting of parkinsonism induced by one or more of the following: Neuroleptics, antipsychotics, lithium, metoclopramide, MDMA, tetrabenazine. Preferably, said secondary parkinsonism is selected from the group consisting of malignant neuroleptic syndrome; drug-induced secondary parkinsonism, secondary parkinsonism due to other external agents; postencephalitic parkinsonism; vascular parkinsonism; and syphilitic parkinsonism.

As used herein, the term “Parkinson's disease” refers to a clinical/pathological condition (e.g., clinical situation) characterized by (e.g., consisting of) the following symptoms: Tremor, bradykinesia, rigidity, postural instability and cognitive impairment. Preferably, said Parkinson's disease comprises one or more of the following: Parkinsonism, Hemiparkinsonism; Paralysis agitans; idiopathic Parkinson's disease; primary Parkinson's disease, and Parkinson's disease dementia.

As used herein, the term “Parkinson-plus syndrome” refers to a clinical/pathological condition (e.g., clinical situation) characterized by the following symptoms: Tremor, bradykinesia, rigidity, postural instability with additional symptoms that distinguish it from Parkinson's disease. Preferably, said Parkinson-plus syndrome is selected from the group consisting of atypical parkinsonism; multiple system atrophy (MSA); progressive supranuclear palsy (PSP); corticobasal degeneration (CBD); dementia with Lewy bodies (DLB); Pick's disease; and olivopontocerebellar atrophy.

These conditions usually lead to the death of dopaminergic neurons of the subject. As used herein, the term “dopaminergic neuron” refers to a neuron that releases dopamine from its synapses. Non-limiting examples of dopaminergic neurons include dopaminergic neurons present in the ventral tegmental area of the midbrain, substantia nigra pars compacta, and arcuate nucleus of the hypothalamus. Dopaminergic neurons may also be tyrosine hydroxylase positive (TH⁺) dopaminergic neurons (e.g., tyrosine hydroxylase is the tyrosine 3-monooxygenase having UniProtKB Accession Number: P07101). Dopaminergic neurons may be obtainable from the mammalian midbrain region, preferably said midbrain region is a substantia nigra, further preferably said midbrain region is pars compacta portion of substantia nigra; most preferably said dopaminergic neurons are obtainable from said midbrain region of a subject (e.g., human) diagnosed with a parkinsonian condition selected from the group consisting of Parkinson's disease, Parkinsonism, RBD and Parkinson-plus syndrome.

As used herein, the term “Rapid Eye Movement (REM) Sleep Behavior Disorder (RBD)” refers to a sleep disorder in which one physically acts out vivid, often unpleasant dreams with vocal sounds and sudden, often violent arm and leg movements during REM sleep—sometimes called dream-enacting behavior. Symptoms of REM sleep behavior disorder may include: Movement, such as kicking, punching, arm flailing or jumping from bed, in response to action-filled or violent dreams, such as being chased or defending yourself from an attack; noises, such as talking, laughing, shouting, emotional outcries or even cursing; and being able to recall the dream if you awaken during the episode. Thus, the major feature of RBD is the loss of muscle atonia (i.e., the loss of paralysis) during otherwise intact REM sleep (during which paralysis is not only normal, but necessary). REM sleep is the stage of sleep in which most vivid dreaming occurs. The loss of motor inhibition leads to a wide spectrum of behavioral release during sleep. Rapid Eye Movement (REM) Sleep Behavior Disorder (RBD) is a very strong predictor or indicator of progression to a synucleinopathy, including Parkinson's disease or dementia with Lewy bodies.

In the context of the present invention, the term “2-hydroxypyridine” means a compound, being of the general formula as given below:

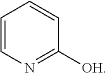

However, the term “2-hydroxypyridine” as used in the context of the present invention also includes an isomer of said 2-hydroxy-pyridine compound or a mixture of isomers thereof; a tautomer of said 2-hydroxypyridine compound or a mixture of tautomers thereof, a derivative of said 2-hydroxypyridine compound or a mixture of derivatives thereof, a pharmaceutically acceptable salt of said 2-hydroxypyridine compound or a mixture of pharmaceutically acceptable salts thereof; or a complex of said 2-hydroxypyridine compound or a mixture of complexes thereof. The term “2-hydroxypyridine” as used within the context of the present invention can be used synonymously to the terms 1,2-dihydro-2-oxopyridine, 1H-2-pyridone, 1H-pyridin-2-one, 2(1H)-PYRIDONE, 2-HYDROXYPYRIDINE, 2-oxopyridine, 2-PYRIDINOL, 2-pyridinone, 2-PYRIDOL, 2-PYRIDON, 2-PYRIDONE, alpha-hydroxypyridine, A-PYRIDONE, AURORA KA-3075, PYRIDIN-2-OL, TIMTEC-BB SBB004392, 1-hydroxy-2-pyridine, 2(1H)pyridinone, alpha-pyridone and pyridone-2.

As used herein, the term “isomer” refers to a compound having the same molecular formula, but differing in the nature or order of bonding of its atoms or in the spatial arrangement of its atoms. Isomers that differ in the arrangement of their atoms in space are referred to as “stereoisomers”. “Stereoisomer” refers to a compound that exists in a different stereoisomeric form if one or more asymmetric centers or asymmetrically substituted double bonds are present and thus can be produced as individual stereoisomers or mixtures. Stereoisomers include enantiomers and diastereomers. Stereoisomers that are not mirror images of one another are termed “diastereomers” and stereoisomers that are non-superimposable mirror images of one another are termed “enantiomers”.

As used herein, the term “tautomer” refers to a specific chemical isomerism characterized by facile interconversion of isomeric forms in equilibrium, especially by migration of a hydrogen atom. In a preferred embodiment of the present invention, a tautomer of 2-hydroxypyridine is 2-pyridone.

As used herein, the term “derivative” refers to a compound or chemical substance related structurally to another substance and theoretically derivable from it, preferably, in the context of the present invention, a compound or chemical substance that is derived from 2-HP as described herein and includes, but is not limited to, amide, ether, ester, amino, carboxyl, acetyl, and/or alcohol derivatives of the 2-HP. More preferably, a derivative of 2-HP according to the present invention may be a moiety of a ring-fused 2-pyridone molecule, most preferably a moiety of FN075. A derivative of 2-HP according to the present invention may also be any pesticide degradation product leading to the formation of 2-HP, most preferably any degradation product of chlorpyrifos, which may be specifically chlorpyrifos oxon, diethyl phosphate, 3,5,6-trichloropyridin-2-ol, diethyl thiophosphate, phosphorothioic acid, ethanol, 3,5,6-trichloro-2-methoxy-pyridine, 2,3-dihydroxypyridine, 2,5-dihydroxypyridine, 2,5,6-trihydroxypyridine, maleamide semialdehyde, maleamic acid, pyruvic acid, aliphatic amines, carbon fragments, carboxyl, NaCl, CO₂ or NH₄CO₃. In one also most preferred embodiment, the derivative of 2-HP is a pesticide degradation product selected from the group consisting of chlorpyrifos oxon, 3,5,6-trichloropyridin-2-ol, 3,5,6-trichloro-2-methoxy-pyridine, 2,3-dihydroxypyridine, 2,5-di hydroxypyridine and 2,5,6-trihydroxypyridine. Chlorpyrifos is an organophosphate pesticide with the chemical structure or formula

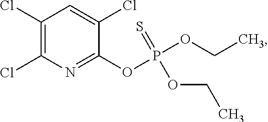 [SMILES: CCOP(=S)(OCC)Oc1nc(Cl)c(Cl)cc1Cl]

used on crops, animals, buildings and in other settings, to kill a number of pests, including insects and worms. It acts on the nervous systems of insects, by inhibiting the acetylcholinesterase enzyme. Other names for chlorpyrifos are: O,O-Diethyl-O-3,5,6-trichloropyridin-2-yl phosphorothioate, Brodan, Bolton insecticide, Chlorpyrifos-ethyl, Cobalt, Detmol UA, Dowco 179, Dursban, Empire, Eradex, Hatchet, Lorsban, Nufos, Pageant, Piridane, Scout, Stipend, Tricel or Warhawk.

The derivative of 2-hydroxypyridine can also be an archaeal cofactor (see Example 5), which is specifically a (bio)chemical compound and/or an ion, important for the catalysis of a biochemical reaction or important for metabolic reactions in a cell.

As used herein, the term “complex” refers to a molecular entity formed by loose association involving two or more component molecular entities (e.g., ionic or uncharged), or the corresponding chemical species, also meaning a chemical association of two or more species (such as ions or molecules), joined usually by weak electrostatic bonds rather than covalent bonds. It is preferred that a complex of 2-HP according to the present invention includes any complex of a moiety of a ring-fused 2-pyridone molecule, most preferably any complex of a moiety of FN075. A complex of 2-HP according to the present invention may be preferably a complex of any pesticide degradation product leading to the formation of 2-HP, most preferably any degradation product of chlorpyrifos, which may be specifically chlorpyrifos oxon, diethyl phosphate, 3,5,6-trichloropyridin-2-ol, diethyl thiophosphate, phosphorothioic acid, ethanol, 3,5,6-trichloro-2-methoxy-pyridine, 2,3-dihydroxypyridine, 2,5-dihydroxpyridine, 2,5,6-trihydroxypyridine, maleamide semialdehyde, maleamic acid, pyruvic acid, aliphatic amines, carbon fragments, carboxyl, NaCl, CO₂ or NH₄CO₃. In one also most preferred embodiment, the complex of 2-HP is a complex of a pesticide degradation product selected from the group consisting of chlorpyrifos oxon, 3,5,6-trichloropyridin-2-ol, 3,5,6-trichloro-2-methoxy-pyridine, 2,3-dihydroxypyridine, 2,5-dihydroxypyridine and 2,5,6-trihydroxypyridine.

The complex of 2-hydroxypyridine can also be a complex of an archaeal cofactor, which is specifically a (bio)chemical compound important for the catalysis of a biochemical reaction or important for metabolic reactions in a cell.

In the context of the present invention, the term “to be at risk of having” is synonymous to “to be at risk of developing”.

The methods of the invention may have diagnostic and prognostic value and this is included within the definition of the term “determining whether or not a subject has a parkinsonian condition” and “determined to have or to be at risk of having the parkinsonian condition”.

As used herein, in the context of the present invention, the term “determined concentration of 2-hydroxypyridine is increased compared to a control” or “determining (a/the) concentration of 2-hydroxypyridine is increased compared to a control, means that the determined concentration of 2-hydroxypyridine in the sample obtained from the subject may be increased by at least 1%, 2%, 3%, 4%, 5%, 6%, 7%, 8%, 9%, 10%, 11%, 12%, 13%, 14% 15% 16% 17% 18% 19% 20% 30% 40%, 50% 60% 70% 80% 90% 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the 2-hydroxypyridine in the control. In the context of the present invention, if the value of the determined concentration of 2-hydroxypyridine is higher, e.g. in a mathematical sense, than the value of the determined concentration of 2-hydroxypyridine of the control, the criterion to have an increase or to be increased compared to the control applies, is present or is justified.

In the context of the present invention, the term “concentration” may be used synonymously with the term “level” or “value”.

In the context of the present invention, the term “control” means a healthy control, preferably a healthy human or an animal, wherein the animal may be an ape, a dog, a cat, a cow, a pig, a horse, a camel, a dromedary, a mouse, a rat, a rabbit, a sheep or a goat. In a more preferred embodiment of the method according to the present invention, the control is a healthy subject. In an even more preferred embodiment of the present invention, the “control” may be a human or patient, who has not been diagnosed with a parkinsonian condition or a human or patient that is not suspected of having, or is at risk of having or is at risk of developing a parkinsonian condition.

In a preferred embodiment of this method, the sample is a biological sample. For example, the concentration or level of 2-HP is measured in such a biological sample by using metabolomic approaches. As used herein, in the context of the present invention, the term “biological sample” includes any body fluid, for example, blood, interstitial fluid, dermal fluid, sweat, and tears. The term “blood” in the context of the invention includes whole blood and its cell-free components, such as, plasma and serum. In a more preferred embodiment, the biological sample is a faecal sample. As used in the context of the present invention, the term “faecal sample” is synonymous to “feces sample”, “fecal sample” or “stool sample”, consisting of, contained in, or relating to the solid waste passed out of the body of a human or animal through the bowels. Thus, the method may be performed by analysing faecal samples. For example, the concentration or level of 2-HP may also be measured in such a faecal sample by using metabolomic approaches.

It is also preferred for the method for determining whether or not a subject has a parkinsonian condition that the concentration of 2-hydroxypyridine is determined by mass spectrometry or NMR.

The term “mass spectrometry”, as used in the context of the present invention, includes any method known to the person skilled in the art, wherein the mass as a value can be determined. This may be, for example, a device including an ion source for generating mainly molecular or pseudo-molecular ions. The ion source may be an atmospheric pressure ionization source, e.g. an Electrospray Ionisation (“ESI”)-ion source, a chemical-atmospheric pressure ionization (“APCI”)-ion source, an Atmospheric Pressure Photoionization (“APPI”)-ion source or an atmospheric pressure ion source. Alternatively, the ion source may comprise a non-atmospheric pressure ionization source, for example, a Fast Atom Bombardment (“FAB”)-ion source, a Liquid Secondary Ion Mass Spectrometry (“LSIMS”)-ion source, a matrix-assisted-laser desorption ionization (“MALDI”)-ion source, a matrix assisted Laser Desorption Ionisation-(“MALDI”)-ion source in combination with a collision cell for collision cooling ions or a Laser Desorption Ionisation (“LDI”)-ion source.

The term “NMR”, as used in the context of the present invention, includes any method known to the person skilled in the art using an NMR apparatus. Such may contain in a statoric frame magnetic means, radio frequency (RF) means comprising excitation circuits, an emitting coil and RF receiving means. Located in a central hole of this statoric frame, this apparatus contains positioning means for holding the sample or the object to analyze. In some cases, especially in solid state NMR, the means for holding the sample have means for spinning it, and sometimes extra means for tilting the spinning axis. The spinning part is called the rotor. It has a cylindrical shape and wears at one end tiny turbo blades driven by air jets. The rotor is made of a material transparent for magnetic fields, generally made of ceramics. It may be filled with the sample to analyze, but when this sample is small, it wears an internal sample container, mechanically centered and whose axis is parallel to the mechanical axis of the rotor. It is necessary to underline that frequently, in NMR literature, the sample container is abusively called in short “sample” instead of “sample container”. As known for many years, in NMR spectroscopy and/or imaging, the sample—be it an object or a subject—is placed inside a strong static and very homogeneous magnetic field B₀. In a quantum description, nuclear spins (assuming that they have a quantum number I=½) can be parallel or anti-parallel with respect to the static magnetic field B₀. Each of these two states has a different energy in the presence of B₀. These energy levels are named Zeeman energy levels, and the spins can absorb energy in the radio-frequency range, to undergo transitions between their two states. In a classical description, the magnetic moments of the nuclear spins process around the static magnetic field. The frequency ω_{L} of the precession (called “Larmor precession”) is roughly proportional to the static magnetic field, also depending on the local chemical environment and can be used to probe molecular structure and dynamics. In order to absorb energy and induce transitions, an oscillating magnetic field B₁ needs to be applied. This field is produced by antennas (i.e. coils) surrounding the object or subject. This field is oscillating at the Larmor frequency (resonance condition) and can be applied for time delays long enough to perturb the magnetization and rotate it at various angles. The NMR method used for determining the specific concentration of 2-hydroxypyridine may include conducting an ¹H-NMR, HSQCs, TOCSY, COSY high-pressure-NMR-spectrum or any further NMR-experiment, known to a person skilled in the art for determining concentrations.

It is also preferred for the methods of the present invention, that the concentration of 2-HP is determined by using a metabolomics approach. This may be done, for example, by extracting polar metabolites, adding 500 μL of MilliQ water to 50 mg fecal matter. Samples are then homogenized using Precellys24 homogenizer (Bertin Technologies) with the settings 6000 rpm, 1×30 sec at 0 to 5° C. Further sample preparation, measurement parameters and data analysis are described in Glaab et. al. (2019).

In a more preferred embodiment, untargeted Gas Chromatography-Mass Spectrometry (GC-MS) with Electron Impact ionization (EI) source could be used for determining the concentration of 2-HR. The result thereof is to identify, validate and quantify 2-HR. The analysis by GC-MS produces a mass intensity spectrum, wherein the peaks of it represent various components of said sample, each component having a characteristic mass-to-charge ratio (m/z) and retention time (RT). The peak representing 2-HP may be compared to a corresponding peak from another spectrum, e.g., from the control sample as defined herein, to obtain a relative measurement. A normalization technique (analytical standard) is used when a quantitative measurement is desired. Such, may for example be carried out like described in the following: Normalizing is achieved by an internal standard, i.e. a ¹³C-labeled compound that differs from endogenous compounds to normalize the data and to evaluate the GC-MS run performance. To enable a quantitative measurement, one has to perform a calibration curve, i.e. additional samples in the GC-MS data acquisition run with increasing concentrations of 2-HR. The concentration of 2-HP is thereby calculated upon linear regression based on (1) the linear function of the calibration curve and (2) the measured value of 2-HP in the sample(s) of interest.

In a further preferred embodiment of this method, the control is determined by measuring the concentration of 2-hydroxypyridine in samples obtained from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, more preferably at least 60, at least 70, at least 80, at least 90 or even more preferably at least 100, 200, 300, 400, 500, 600, 700, 800, 900, or 1000 control subjects, wherein the control subjects are healthy control subjects characterized by not having the parkinsonian condition. In a further preferred embodiment of this method, the control is determined by measuring the concentration of 2-hydroxypyridine in samples obtained from at least 45, preferably at least 50, more preferably at least 100, control subjects, wherein the control subjects are healthy control subjects characterized by not having the parkinsonian condition. Thus, in one preferred embodiment, the control is obtained by measuring the same marker in a set of at least 100 control subjects, wherein a control subject is a healthy control subject characterized by not having the parkinsonian condition. In a further preferred embodiment of this method, the control is determined by measuring the concentration of 2-hydroxypyridine in samples obtained from at least 1000, 2000, 3000, 4000, 5000, 6000, 7000, 8000, 9000 or even 10000 control subjects, wherein the control subjects are healthy control subjects characterized by not having the parkinsonian condition. Depending upon the difference between the determined concentration of 2-hydroxypyridine in the sample obtained from the subject and the control, the subject can be diagnosed as having, being at risk of having, being predisposed to develop a parkinsonian condition, or as not having a parkinsonian condition.

It is also possible, that the method further comprises determining the concentration of beta-glutamic acid, conducting a neurological test or classification according to Unified Parkinson's Disease Rating Scale (UPDRS).

The term “neurological test” as used in the context of the present invention means any test for assessing a person's neurological function. Such a test should be designed to evaluate sensory skills, motor skills, speech, hearing, vision, balance, coordination and mood. A neurological examination is used to diagnose a wide range of neurological disorders. A neurological test is usually carried out in several separate stages, each of which tests different nervous system functions. In the mental status portion of the test, for example, the patient will answer a number of questions that examines their mood and thoughts, their awareness, and facets of their intellectual capacity, such as speech, language, memory and judgement. During this part of the test, the patient's behaviour is also being noted for particular types of emotional or behavioural responses. Other parts of the neurological test examine the patient's nervous, motor and sensory systems. One of the most important is the cranial nerves test, which investigates physical functions such as peripheral vision, the gag reflex, smell and taste, and sensation in the head and neck area. In the motor system test, the patient's muscle function is examined for signs of atrophy or abnormal movements, which might indicate abnormalities. To examine the sensory system, the responses to pain, pressure, temperature, and other stimuli is investigated. This part of the neurological test is usually repeated at least once to ensure that the results are accurate. Reflexes and coordination are tested in a similar fashion. In coordination tests, for example, the patient is asked to move their fingers or other parts of the body in various ways, while noting how well the patient can carry out these requests. During a neurological test, it is crucial that the person being tested is as accurate as possible with their responses to test questions.

The term “Movement Disorder Society-Unified Parkinson's Disease Rating Scale (MDS-UPDRS)” as used in the context of the present invention means a comprehensive 50 question assessment of both motor and non-motor symptoms associated with Parkinson's disease.

Thus, in a preferred embodiment of the method for determining whether or not a subject has a parkinsonian condition, the concentration of 2-HP will be measured in combination with other signs, symptoms and clinical tests of a parkinsonian condition. Other signs and symptoms of a parkinsonian condition may include wearing off and dyskinesia, hallucinations or delusions, impulsive and compulsive behaviour or problems sleeping.

It may also be preferred that the subject is a human.

The present invention also relates to a method for monitoring the progression of a parkinsonian condition in a subject diagnosed with the parkinsonian condition, comprising (i) determining the concentration of 2-hydroxypyridine in a sample obtained from the subject, wherein the determined concentration of 2-hydroxypyridine is compared to a control sample of the same subject.

The same definitions as given above herein also apply to this method for monitoring the progression of a parkinsonian condition.

Thus, the invention provides a method for monitoring a PD patient over time to determine whether the disease is progressing.

The method may be performed by analysing faecal samples, from a subject at a certain time; determining the concentration of 2-HP; and comparing the determined concentration with the one measured with respect to a biological sample obtained from the same subject at an earlier time. Depending upon the difference between the determined 2-HP concentrations, it can be seen whether the biomarker level has increased, decreased or remained constant over the respective interval.

It is preferred for said method for monitoring the progression of a parkinsonian condition that the control sample has been obtained from the subject a certain time before step (i), more preferably wherein certain time means at least 1, 2, 3, 4, 5, 6, 7, 8, 9, 10 or 11 months, even more preferably at least 1 year, 2 years, 3 years, 4 years, 5 years, 6 years, 7 years, 8 years, 9 years or even 10 years, before step (i).

It is also preferred for said method for monitoring the progression of a parkinsonian condition that the control sample has been obtained from the subject a certain time before step (i), more preferably wherein certain time means at least 6 months, even more preferably at least 1 year, before step (i).

In a preferred embodiment of said method for monitoring the progression of a parkinsonian condition, the progression of the parkinsonian condition is positive, if the concentration of 2-hydroxypyridine has decreased compared to the control sample of the same subject or has maintained the same compared to the control sample of the same subject. The term “progression of the parkinsonian condition is positive” means in the context of the present invention that an improvement of the disease status of a/the subject or patient has occurred, e.g. the parkinsonian condition is lower or not that much present, distinct or strong anymore.

The concentration of 2-hydroxypyridine has decreased compared to the control sample of the same subject means, in the context of the present invention and for the method of monitoring the progression of a parkinsonian condition, that the concentration of 2-hydroxypyridine in the sample of the subject has decreased by at least 11%, 12%, 13%, 14%, 15% 16% 17% 18% 19% 20% 30% 40% 50% 60% 70% 80% 90% 100% 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample.

The concentration of 2-hydroxypyridine has maintained the same compared to the control sample of the same subject means that the concentrations only deviate by +10% or −10%.

It is also preferred for said method for monitoring the progression of a parkinsonian condition that the progression of the parkinsonian condition is negative, if the concentration of 2-hydroxypyridine has increased compared to the control sample of the same subject, more preferably wherein the concentration of 2-hydroxypyridine has significantly increased compared to the control sample of the same subject. The term “progression of the parkinsonian condition is negative” means in the context of the present invention that a worsening of the disease status of a/the subject or patient has occurred, e.g. the parkinsonian condition is much more present, distinct, strong or evident.

The concentration of 2-hydroxypyridine has increased compared to the control sample of the same subject means, in the context of the present invention and for the method for monitoring the progression of a parkinsonian condition, that the concentration of 2-hydroxypyridine in the sample of the subject has increased by at least 1%, 2%, 3%, 4%, 5%, 6%, 7%, 8%, 9%, 10%, 11%, 12%, 13%, 14%, 15%, 16%, 17%, 18%, 19%, 20% 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample. In the context of the present invention and as used for this embodiment, if the value of the determined concentration of 2-hydroxypyridine is higher, e.g. in a mathematical sense, than the value of the determined concentration of 2-hydroxypyridine of the control sample from the same subject, the criterion to have an increase or to be increased compared to the control applies, is present or is justified.

A significant increase in this embodiment is given, when the concentration of 2-hydroxypyridine in the sample of the subject has increased by at least 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample. In a more preferred embodiment, a significant increase is given, when the concentration of 2-hydroxypyridine in the sample of the subject has increased by at least 30%, compared to the concentration of 2-hydroxypyridine in the control sample.

The present invention also relates to a method for assessing the efficacy of treatment of a parkinsonian condition in a subject diagnosed with the parkinsonian condition, comprising (i) determining the concentration of 2-hydroxypyridine in a sample obtained from the subject, wherein the determined concentration of 2-hydroxypyridine is compared to a control sample of the same subject.

The same definitions as given above herein also apply to this method for assessing the efficacy of treatment of a parkinsonian condition.

Thus, the invention provides a method for assessing the efficacy of treatment of a PD patient over time to determine whether the treatment is successful or efficient.

The method may also be performed by analysing faecal samples, from a subject at a certain time; determining the concentration of 2-HP; and comparing the determined concentration with the one measured with respect to a biological sample obtained from the same subject at an earlier time. Depending upon the difference between the determined 2-HP concentrations, it can be seen whether the course of treatment should be maintained, changed or adapted.

It is preferred for said method for assessing the efficacy of treatment of a parkinsonian condition that the control sample has been obtained from the subject a certain time before step (i), more preferably wherein certain time means at least 1, 2, 3, 4, 5, 6, 7, 8, 9, 10 or 11 months, even more preferably at least 1 year, 2 years, 3 years, 4 years, 5 years, 6 years, 7 years, 8 years, 9 years or even 10 years, before step (i).

It is also preferred for said method that the control sample has been obtained from the subject a certain time before step (i), more preferably wherein certain time means at least 6 months, even more preferably at least 1 year, before step (i).

In a preferred embodiment of said method for assessing the efficacy of treatment of a parkinsonian condition, the efficacy of treatment of the parkinsonian condition is assessed as being positive, if the concentration of 2-hydroxypyridine has decreased compared to the control sample of the same subject or has maintained the same compared to the control sample of the same subject. The term “efficacy of treatment of the parkinsonian condition is assessed as being positive” means in the context of the present invention and for the method for assessing the efficacy of treatment of a parkinsonian condition, that an improvement of the disease status of a/the subject or patient has occurred, e.g. the parkinsonian condition is lower or not that much present, distinct or strong anymore.

The concentration of 2-hydroxypyridine has decreased compared to the control sample of the same subject means, in the context of said method, that the concentration of 2-hydroxypyridine in the sample of the subject has decreased by at least 11%, 12%, 13%, 14%, 15% 16% 17% 18% 19% 20% 30% 40%, 50% 60% 70% 80% 90% 100% 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample.

The concentration of 2-hydroxypyridine has maintained the same compared to the control sample of the same subject means that the concentrations only deviate by +10% or −10%.

It is also preferred for said method for assessing the efficacy of treatment of a parkinsonian condition, that the efficacy of treatment of the parkinsonian condition is assessed as being negative, if the concentration of 2-hydroxypyridine has increased compared to the control sample of the same subject, more preferably wherein the concentration of 2-hydroxypyridine has significantly increased compared to the control sample of the same subject. The term “efficacy of treatment of the parkinsonian condition is assessed as being negative” means in the context of the present invention that a worsening of the disease status of a/the subject or patient has occurred, e.g. the parkinsonian condition is much more present, distinct, strong or evident.

The concentration of 2-hydroxypyridine has increased compared to the control sample of the same subject means, in the context of the present invention and for the method for assessing the efficacy of treatment of a parkinsonian condition, that the concentration of 2-hydroxypyridine in the sample of the subject has increased by at least 1%, 2%, 3%, 4%, 5%, 6%, 7%, 8%, 9%, 10%, 11%, 12%, 13%, 14%, 15%, 16%, 17%, 18%, 19%, 20% 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample. In the context of the present invention and as used for this embodiment, if the value of the determined concentration of 2-hydroxypyridine is higher, e.g. in a mathematical sense, than the value of the determined concentration of 2-hydroxypyridine of the control sample from the same subject, the criterion to have an increase or to be increased compared to the control applies, is present or is justified.

A significant increase in this embodiment is given, when the concentration of 2-hydroxypyridine in the sample of the subject has increased by at least 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample. In a more preferred embodiment, a significant increase is given, when the concentration of 2-hydroxypyridine in the sample of the subject has increased by at least 30%, compared to the concentration of 2-hydroxypyridine in the control sample.

The present invention also relates to a kit-of-parts for determining whether or not a subject has a parkinsonian condition, comprising means for determining the concentration of 2-hydroxypyridine in a sample obtained from the subject and a test system that is capable of determining whether or not said subject has a parkinsonian condition, based on the result of the determined concentration of 2-hydroxypyridine of said subject compared to a control. Thus, the test system includes establishing a control, e.g. as described above for the method of determining whether or not a subject has a parkinsonian condition. For the kit-of-parts and the test system, it may be determined that the subject has a parkinsonian condition if the concentration of 2-hydroxypyridine has increased compared to the control or if the value of the concentration of 2-hydroxypyridine lies above the value of the control, more preferably wherein the concentration of 2-hydroxypyridine has significantly increased compared to the control or if the value of the concentration of 2-hydroxypyridine lies significantly above the value of the control.

The concentration of 2-hydroxypyridine has increased compared to the control means for this embodiment, that the concentration of 2-hydroxypyridine in the sample of the subject has increased by at least 1%, 2%, 3%, 4%, 5%, 6%, 7%, 8%, 9%, 10%, 11%, 12%, 13% 14% 15% 16% 17% 18% 19% 20% 30% 40% 50% 60% 70% 80% 90% 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control. In the context of the present invention and as used for this embodiment, if the value of the determined concentration of 2-hydroxypyridine is higher, e.g. in a mathematical sense, than the value of the determined concentration of 2-hydroxypyridine of the control, the criterion to have an increase or to be increased compared to the control applies, is present or is justified.

It is preferred for the kit-of-parts and the test system, that it is determined that the subject not has a parkinsonian condition, if the concentration of 2-hydroxypyridine has decreased compared to the control or if the value of the concentration of 2-hydroxypyridine lies below the value of the control. The definitions for the term “decrease” or “decreased” as given above also apply for this embodiment.

In some aspects, the present invention relates to the kit-of-parts as described herein, wherein the kit-of-parts comprises means for determining the concentration of 2-hydroxypyridine, isomer, tautomer, derivative, pharmaceutically accepted salt, complex or mixtures thereof as described herein.

These means for determining the concentration of 2-hydroxypyridine in a sample obtained from the subject may be or may comprise sample collection, GC-MS measurements and 2-HP quantification known to a person skilled in the art.

Unless otherwise specified, the terms used herein have their common general meaning as known in the art.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES OF THE INVENTION

In order that the invention may be readily understood and put into practical effect, some aspects of the invention are described by way of the following non-limiting examples.

Supplementary Methods:

Stool Extraction and GC-MS Measurement

Stool samples were collected and snap-frozen in liquid nitrogen without any additives or stabilizers. Storage was performed within 1 h post-collection in −80° C. and shipment was on dry ice. Stool samples were homogenized using a cryogenic grinder (6875D Freezer/Mill®-SPEX). To extract polar metabolites, 500 μL of MilliQ water was added to 50 mg fecal matter. Then, samples were homogenized using Precellys24 homogenizer (Bertin Technologies): 6000 rpm, 1×30 sec at 0 to 5° C. Then, untargeted GC-MS analysis (Quadrupole, EI Source) was performed for the identification, validation and quantification by external calibration measurements with analytical standards (Sigma Aldrich, 41152). Further sample preparation, measurement parameters and data analysis were described in Glaab et al. (2019).

### Example 1: Alpha-Synuclein-Yeast Experiment

Yeast strains, which have been used, are shown in Table 1 below.

TABLE 1

| | | |
|---|---|---|
| W303 | MATa ura3-52 trp1Δ2 leu2-3_112 his3-11 ade2-1 | Parental |
| | can1-100 | strain |
| HiTox | W303 pdr5Δ::KanMX4, pAG306GAL-SNCA-EGFP, | Derived |
| | pAG304GAL-SNCA-EGFP | strain |
| Control | W303 pdr5Δ::KanMX4, pAG306GAL-ccdB-EGFP, | Derived |
| | pAG304GAL-ccdB-EGFP | strain |

Yeast Cultivation Media

Yeast cells were grown in synthetic complement (SC) media containing 6.7 g/L yeast nitrogen base without amino acids, 5 g/L ammonium sulfate supplemented with 2 g/L SC. Media was autoclaved and the carbon source (8% raffinose or galactose) was added to a final concentration of 2%.

Yeast Cultivation and Phenotyping

Four fresh single colonies of the Hitox strain and its respective control strain were inoculated from SC-2% glucose plates into 5 mL SC-2% raffinose and incubated overnight with shaking (200 rpm) at 30° C. After ≈20 h, overnight cultures were diluted to OD 0.5 and 2 μL of the yeast cultivation was added to 78 μL drug containing media to a final optical density of 0.0125 in a 384-well microplate. 2-HP was diluted in SC-2% raffinose/galactose and tested at different concentrations (1-100 mM). Finally, the plates are measured in a microplate reader (TECAN™ Infinite M200Pro), at an interval of 10 minutes during 72 h at 30° C. Yeast growth phenotyping was performed as previous described (Jung et al. 2015). Final biomass was corrected using the GATHODE software (Jung et al. 2015). The OD₆₀₀ at 48 h was taken for the quantification.

### Example 2: Dopaminergic Neuron Experiment

Enteric Neurons Cultivation and Phenotyping

Enteric neurons were cultured in differentiation medium, consisting on Neurobasal (Invitrogen, 21103049) supplemented with 1:200 N2 (Invitrogen, 17502048), 1:100 L-Glutamine (Invitrogen, 25030-024), 1:100 B-27 without Vitamin A (Life Technologies, 12587-010), 1:100 Penicillin/Streptomycin (Invitrogen, 15140122), 25 ng/mL GDNF (Peprotech, 450-10) and 100 μM Ascorbic Acid (Sigma, A5960). Cells were cultured for 21 days in a 6-well plate, then detached with Accutase (Sigma, A6964), and replated into a 96-well plate. Cells were maintained under differentiating conditions until day 31, when they were treated with increasing concentrations of 2-hydroxypyridine (Sigma, H56800). Tested concentrations were as follows: 1 μM, 3 μM, 6 μM, 10 μM, 30 μM, 60 μM, 100 μM, 300 μM, 600 μM, 1 mM and 3 mM.

2-hydroxypyridine was reconstituted to 10 mM in the differentiation medium described above.

Cells were treated for 24 h, then cell viability was assessed performing a Tetrazolium (MTT) assay. Thiazolyl Blue tetrazolium Bromide (Sigma, M5655) was reconstituted to a 5 mg/mL solution in differentiation media and filtered. 10 μL of the solution were then added to every well containing 100 μL of media. Plate was incubated for 2 h in a normal incubator (37° C., 5% CO₂). Media containing MTT solution was removed and 100 μL of DMSO (Sigma, D8418) were added to lyse the cells. Cells were disaggregated by pipetting vigorously and absorbance was read at 570 nm, using the microplate Cytation 5M reader (Biotek).

Alpha-Synuclein Aggregation Staining in Enteric Neurons

Cells fixed in 4% paraformaldehyde (Merck Millipore, 1004965000) for 15 min at RT and then washed 3× for 5 min with PBS at RT. Prior to immunostaining, a permeabilization step was performed using 0.05% Triton-X100 solution in PBS for 10 min at 4° C. Cells were then blocked for 1 h at RT with 10% FBS in PBS. Incubation with α-synuclein antibody (NOVUS biologicals, NBP1-05194, 1:1000), α-synuclein filament antibody (Abcam, ab20953, 1:5000) and TUJ1 (Millipore, AB9354, 1:600) was done overnight at 4° C. in blocking buffer. The following day, cells were washed with PBS 3× for 5 min at RT. Then, incubation with the corresponding secondary antibodies, Alexa Fluor anti-chicken 488 (Invitrogen, A-11039, 1:1000), Alexa Fluor anti-mouse IgG1 647 (Invitrogen, A-21240, 1:1000) and Alexa Fluor anti-rabbit 568 (Invitrogen, A11036, 1:1000) was performed for 2 h at RT in blocking buffer. Hoechst 33342 solution (Invitrogen, 62249, 1:1000) was added during this step to stain the nuclei. After incubation, cells were washed 3× with PBS and imaged directly afterwards.

### Example 3: In Silico Target Prediction of 2-HP

To identify putative protein targets of 2-HP, an inverse in silico screening was performed. This analysis is based on in silico docking and binding affinity prediction with proteins. In general, these methods have a high sensitivity (recall around 80%) but a lower specificity (precision between 20 and 40%) and require further filtering and in vitro validation to remove false positives.

Multiple putative targets in relation with Parkinson's disease have been identified such as dopamine beta-hydroxylase (DBH) and cathechol o-methyltransferase (COMT) both involved in the dopamine metabolism. For DBH, a crystal structure was available which enabled a stronger docking and binding affinity prediction analysis, hence confirming that 2-HP has at least a weak affinity for this target. In addition, DBH polymorphisms have already been shown in the literature to be tightly linked to PD pathology.

### Example 4: 2-HP Upregulates Proinflammatory Cytokine IL-1b in a Murine Macrophage Model

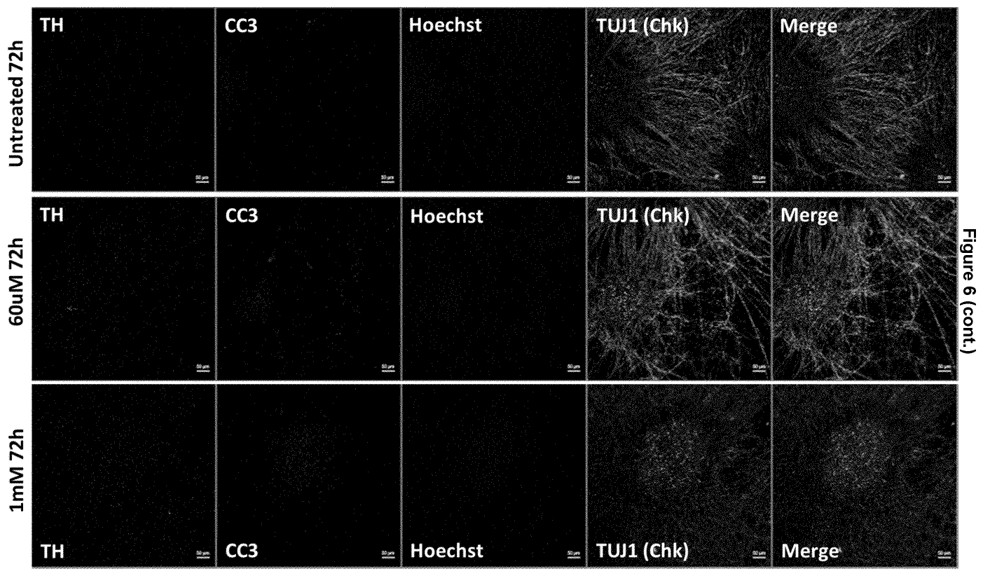

The inventors have tested whether 2-HP can act as signal 1 in the activation of the NLRP3 inflammasome in murine bone-barrow derived macrophages (BMDMs). NLRP3 inflammasome activation is thought to play a role in the pathogenesis of neurodegenerative disorders, and increased levels of this inflammasome and of the related cytokine IL-1b have been detected in Parkinson's patients (Guan & Han, 2020). In these experiments, the cells were grown in BMDM growth media and treated with 2-hydroxypyridine (2.5 and 10 mM) for up to 72 hours. Inflammasome activation was evaluated with qPCR for IL-1b expression (normalized to β-actin), considering the fold change relative to 0 mM 2-HP of each time point. There was a 3.2-fold increase in IL-1b mRNA after 3 hours of 10 mM 2-HP treatment, and 1.5-fold increase after 24 hours (FIG. 7).

### Example 5: Archaeal Enzyme Linked to 2-HP is Present in Sequence Data from Stool Samples

To verify that 5,10-methenyltetrahydromethanopterin hydrogenase, the archaeal enzyme that has a 2-HP derivative cofactor, is present in the MiBiPa stool samples, the inventors have mined metagenomic and metatranscriptomic sequence reads to look for this enzyme. The inventors were able to detect it in the samples, and it was present in a higher proportion of samples from PD and RBD patients than from control subjects (see Table 2).

TABLE 2
Percent (%) of stool samples that contain the archaeal enzyme
5,10-methenyltetrahydromethanopterin hydrogenase.

| Type of data | Ctrl | PD | RBD |
|---|---|---|---|
| Metagenomic | 36.73 | 48.94 | 40.74 |
| Metatranscriptomic | 40.82 | 51.06 | 48.15 |

### Example 6: Genus Methanobrevibacter is More Abundant in PD Compared to Controls in a Large Cohort

As 2-HP levels are higher in PD and RBD patients than in controls, and correlated with Methanobrevibacter abundance, the inventors tested whether Methanobrevibacter is more abundant in PD and RBD patients than in controls. Comparing 16S rRNA gene amplicon data from MiBiPa subjects, there was a numerical difference between groups. Considering similar data from a larger cohort, NCER-PD (Baldini et al. 2020), there was a statistically significant difference between the PD and control groups for this genus (FIG. 8). The difference was also significant if the two cohorts were combined and tested together (p=0.003; fdr=0.015).

### Example 7: 2-HP is Detectable in Archaeal Cell Cultures

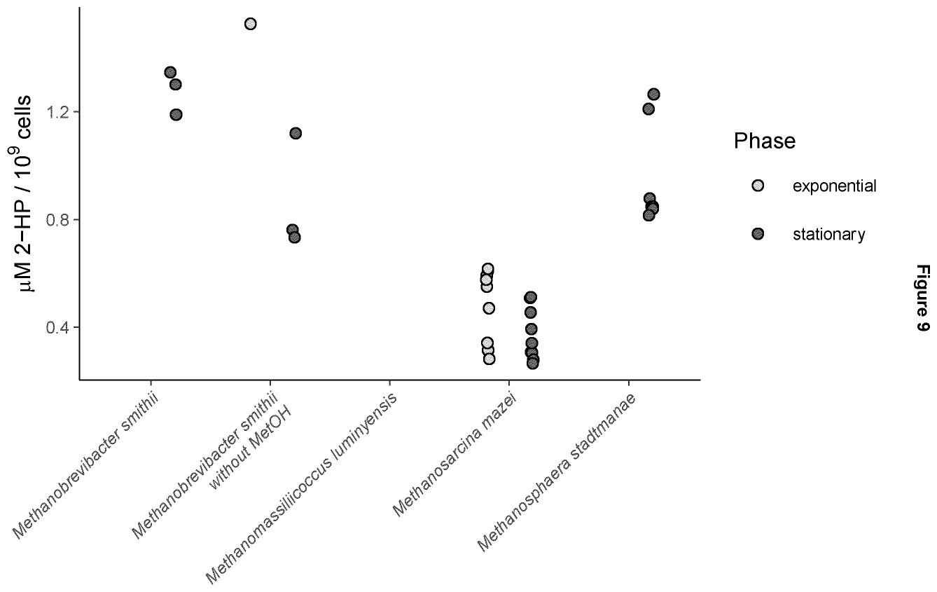

To validate that 2-HP is linked to archaeal metabolism and for showing correlations between 2-HP concentration and Methanobrevibacter abundance in stool samples, the inventors cultured several archaeal species and measured the concentrations of 2-HP in the cultures with targeted GC/MS. 2-HP was detectable in these samples, with the highest cell count normalized concentrations in cell pellets of Methanobrevibacter smithii and Methanosphaera stadtmanae, two archaeal species present in the human gut (FIG. 9).

### Example 8: In Vivo Results Show Sensorimotor Deficits Due to 2-HP Treatment

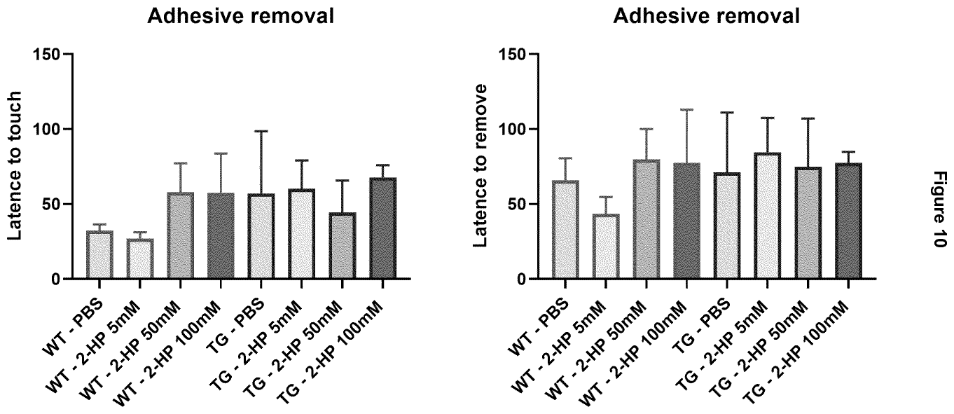

The in vivo work testing for effects of 2-HP in a mouse model lead to results from a small-scale 15-day experiment. In this experiment, 2-HP was injected at several concentrations (5, 50 and 100 mM) into the brain of transgenic mice overexpressing human alpha-synuclein, and of wildtype control mice. Behavioral assays at 15 days post-injection showed that wildtype mice treated with higher 2-HP concentrations perform worse in the adhesive removal test (a measure of sensorimotor function; Fleming et al. 2013) than those treated with control solution (PBS) (FIG. 10).

The present invention further relates to the following items:

1. A method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control.
2. Method according to item 1, wherein the sample is a biological sample.
3. Method according to item 2, wherein the biological sample is a faecal sample.
4. Method according to any one of the preceding items, wherein the concentration of 2-hydroxypyridine is determined by mass spectrometry or NMR.
5. Method according to any one of the preceding items, wherein the control is determined by measuring the concentration of 2-hydroxypyridine in samples obtained from at least 45, preferably at least 100, control subjects, wherein the control subjects are healthy control subjects characterized by not having the parkinsonian condition.
6. Method according to any one of the preceding items, wherein the method further comprises determining the concentration of beta-glutamic acid, conducting a neurological test or classification according to Unified Parkinson's Disease Rating Scale (UPDRS).
7. Method according to any one of the preceding items, wherein the subject is a human.
8. A method for monitoring the progression of a parkinsonian condition in a subject diagnosed with the parkinsonian condition, comprising
(i) determining the concentration of 2-hydroxypyridine in a sample obtained from the subject, wherein the determined concentration of 2-hydroxypyridine is compared to a control sample of the same subject.
9. Method according to item 8, wherein the control sample has been obtained from the subject a certain time before step (i), preferably wherein certain time means at least 6 months, more preferably at least 1 year, before step (i).
10. Method according to item 8 or 9, wherein the progression of the parkinsonian condition is positive, if the concentration of 2-hydroxypyridine has decreased compared to the control sample of the same subject or has maintained the same compared to the control sample of the same subject.
11. Method according to item 8 or 9, wherein the progression of the parkinsonian condition is negative, if the concentration of 2-hydroxypyridine has increased compared to the control sample of the same subject, preferably wherein the concentration of 2-hydroxypyridine has significantly increased compared to the control sample of the same subject.
12. A method for assessing the efficacy of treatment of a parkinsonian condition in a subject diagnosed with the parkinsonian condition, comprising
(i) determining the concentration of 2-hydroxypyridine in a sample obtained from the subject, wherein the determined concentration of 2-hydroxypyridine is compared to a control sample of the same subject.
13. Method according to item 12, wherein the control sample has been obtained from the subject a certain time before step (i), preferably wherein certain time means at least 6 months, more preferably at least 1 year, before step (i).
14. Method according to item 12 or 13, wherein the efficacy of treatment of the parkinsonian condition is assessed as being positive, if the concentration of 2-hydroxypyridine has decreased compared to the control sample of the same subject or has maintained the same compared to the control sample of the same subject.
15. Method according to item 12 or 13, wherein the efficacy of treatment of the parkinsonian condition is assessed as being negative, if the concentration of 2-hydroxypyridine has increased compared to the control sample of the same subject, preferably wherein the concentration of 2-hydroxypyridine has significantly increased compared to the control sample of the same subject.
16. Kit-of-parts for determining whether or not a subject has a parkinsonian condition, comprising means for determining the concentration of 2-hydroxypyridine in a sample obtained from the subject and a test system that is capable of determining whether or not said subject has a parkinsonian condition, based on the result of the determined concentration of 2-hydroxypyridine of said subject compared to a control.

### REFERENCES

Baldini F, Hertel J, Sandt E, et al. Parkinson's disease-associated alterations of the gut microbiome predict disease-relevant changes in metabolic functions. BMC Biol. 2020; 18(1):62. Published 2020 Jun. 9. doi:10.1186/s12915-020-00775-7.
Braak, H., Rüb, U., Gai, W. P., & Del Tredici, K. (2003). Idiopathic Parkinson's disease. Possible routes by which vulnerable neuronal types may be subject to neuroinvasion by an unknown pathogen. Journal of neural transmission (Vienna, Austria: 1996), 110(5), 517-536.
Braak, H., De Vos, R. A. I., Bohl, J., & Del Tredici, K. (2006). Gastric α-synuclein immunoreactive inclusions in Meissner's and Auerbach's plexuses in cases staged for Parkinson's disease-related brain pathology. Neuroscience Letters, 396(1), 67-72.
Cairns, A. G., Vazquez-Romero, A., Mandi Moein, M., Ådén, J., Elmore, CS., Takano, A., Arakawa, R., Varrone, A., Almqvist, F., & Schou, M. (2018). Increased Brain Exposure of an Alpha-Synuclein Fibrillization Modulator by Utilization of an Activated Ester Prodrug Strategy. Chem Neurosci., 9(11), 2542-2547.
Chaudhuri, K. R., & Schapira, A. H., (2009). Non-motor symptoms of Parkinson's disease: dopaminergic pathophysiology and treatment, Lancet Neurol. 8(5), 464-74.
Chermenina, M., Chorell, E., Pokrzywa, M., Antti, H., Almqvist, F., Strömberg, I. & Pernilla Wittung-Stafshede (2015). Single injection of small-molecule amyloid accelerator results in cell death of nigral dopamine neurons in mice. npj Parkinson's Disease, volume 1, Article number: 15024.
Dorsey, E. R., Sherer, T., Okun, M. S., & Bloemd, B. R. (2018). The emerging evidence of the Parkinson pandemic. Journal of Parkinson's Disease, 8(s1), S3-S8.
Fleming S M, Ekhator O R, Ghisays V. Assessment of sensorimotor function in mouse models of Parkinson's disease. J Vis Exp. 2013; (76):50303. Published 2013 Jun. 17. doi:10.3791/50303.
Glaab, E., Trezzi, J.-P., Greuel, A., Jäger, C., Hodak, Z., Drzezga, A., Timmermann, L., Tittgemeyer, M., Diederich, N.J., & Eggers, C. (2019) Integrative analysis of blood metabolomics and PET brain neuroimaging data for Parkinson's disease, Neurobiology of Disease. 124, 555-562.
Guan Y, Han F. Key Mechanisms and Potential Targets of the NLRP3 Inflammasome in Neurodegenerative Diseases. Front Integr Neurosci. 2020; 14:37. Published 2020 Jul. 24. doi:10.3389/fnint.2020.00037.
Harishankar, M. K., Sasikala, & C., Ramya, S. M. (2003). Efficiency of the intestinal bacteria in the degradation of the toxic pesticide, chlorpyrifos. 3 Biotech., 3(2): 137-142.
Heintz-Buschart, A., Pandey, U., Wicke, T., Sixel-Döring, F., Janzen, A., Sittig-Wiegand, E., Trenkwalder, C., Oertel, W. H., Mollenhauer, B., Wilmes, P.° (2018) The nasal and gut microbiome in Parkinson's disease and idiopathic rapid eye movement sleep behavior disorder. Movement Disorders 33, 88-98.
Horvath, I., Weise C. F., Andersson E. K., Chorell, E., Sellstedt, M., Bengtsson, C., Ol-ofsson, A., Hultgre, S. J., Chapman, M., Wolf-Watz, M., Almqvist, F., & Wittung-Stafshede, P. (2012). Mechanisms of Protein Oligomerization: Inhibitor of Functional Amyloids Templates α-Synuclein Fibrillation, J. Am. Chem. Soc., 134, 7, 3439-3444.
Jung, P. P, Christian, N., Kay, D. P., Skupin, A., & Linster, C. L. (2015). Protocols and programs for high-throughput growth and aging phenotyping in yeast, PLoS One, 10(3).
Keshavarzian, A., Green, S. J., Engen, P. A., Voigt, R. M., Naqib, A., Forsyth, C. B., . . . Shannon, K. M. (2015). Colonic bacterial composition in Parkinson's disease. Movement Disorders, 30(10), 1351-1360.
Olanow, C. W., & Kieburtz K. (2010). Defining disease-modifying therapies for PD-A road map for moving forward. Mov Disord., 25(12):1774-9.
Scheperjans, F., Aho, V., Pereira, P. A. B., Koskinen, K., Paulin, L., Pekkonen, E., . . . Auvinen, P. (2015). Gut microbiota are related to Parkinson's disease and clinical phenotype. Movement Disorders, 30(3), 350-358.
Scheperjans, F., Pekkonen, E., Kaakkola, S., & Auvinen, P. (2015). Linking Smoking, Coffee, Urate, and Parkinson's Disease—A Role for Gut Microbiota?. J Parkinsons Dis., 5(2):255-62.
Schrag, A., Horsfall, L., Walters, K., Noyce, A., Petersen, I. (2015). Prediagnostic presentations of Parkinson's disease in primary care: a case-control study. Lancet Neurol., 14(1):57-64.
Simonsen, A. H., Kuiperij, B., El-Agnaf, O. M., Engelborghs, S., Herukka, S. K., Parnetti, L., Rektorova, I., Vanmechelen, E., Kapaki, E., Verbeek, M., & Mollenhauer, B. (2016). The utility of α-synuclein as biofluid marker in neurodegenerative diseases: a systematic review of the literature. Biomark Med., 10(1):19-34.
Vogt S., Lyon E. J., Shima S., and Thauer R. K. (2008). The exchange activities of [Fe] hydrogenase (iron-sulfur-cluster-free hydrogenase) from methanogenic archaea in comparison with the exchange activities of [FeFe] and [NiFe] hydrogenases. J Biol Inorg Chem, 13(1): 97.
Mollenhauer, B., Zimmermann, J., Sixel-Döring, F., Focke, N. K., Wicke, T., Ebentheuer, J., Schaumburg, M., Lang, E., Trautmann, E., Zetterberg, H., Taylor, P., Friede, T., & Trenkwalder, C. DeNoPa Study Group. (2016). Monitoring of 30 marker candidates in early Parkinson disease as progression markers. Neurology, 87(2):168-77.

1. A method for determining whether or not a subject has a parkinsonian condition, comprising determining the concentration of 2-hydroxypyridine in a sample obtained from the subject, wherein the subject is determined to have, or to be at risk of having the parkinsonian condition, if the determined concentration of 2-hydroxypyridine is increased compared to a control.

2. Method according to claim 1, wherein the sample is a biological sample.

3. Method according to claim 2, wherein the biological sample is a faecal sample.

4. Method according to any one of the preceding claims, wherein the concentration of 2-hydroxypyridine is determined by mass spectrometry or NMR.

5. Method according to any one of the preceding claims, wherein the control is determined by measuring the concentration of 2-hydroxypyridine in samples obtained from at least 45, preferably at least 100, control subjects, wherein the control subjects are healthy control subjects characterized by not having the parkinsonian condition.

6. Method according to any one of the preceding claims, wherein the method further comprises determining the concentration of beta-glutamic acid, conducting a neurological test or classification according to Unified Parkinson's Disease Rating Scale (UPDRS).

7. Method according to any one of the preceding claims, wherein the subject is a human.

8. A method for monitoring the progression of a parkinsonian condition in a subject diagnosed with the parkinsonian condition, comprising
(i) determining the concentration of 2-hydroxypyridine in a sample obtained from the subject, wherein the determined concentration of 2-hydroxypyridine is compared to a control sample of the same subject.

9. Method according to claim 8, wherein the control sample has been obtained from the subject a certain time before step (i), preferably wherein certain time means at least 6 months, more preferably at least 1 year, before step (i).

10. Method according to claim 8 or 9, wherein the progression of the parkinsonian condition is positive, if the concentration of 2-hydroxypyridine has decreased compared to the control sample of the same subject or has maintained the same compared to the control sample of the same subject.

11. Method according to claim 8 or 9, wherein the progression of the parkinsonian condition is negative, if the concentration of 2-hydroxypyridine has increased compared to the control sample of the same subject, preferably wherein the concentration of 2-hydroxypyridine has significantly increased compared to the control sample of the same subject.

12. A method for assessing the efficacy of treatment of a parkinsonian condition in a subject diagnosed with the parkinsonian condition, comprising
(i) determining the concentration of 2-hydroxypyridine in a sample obtained from the subject, wherein the determined concentration of 2-hydroxypyridine is compared to a control sample of the same subject.

13. Method according to claim 12, wherein the control sample has been obtained from the subject a certain time before step (i), preferably wherein certain time means at least 6 months, more preferably at least 1 year, before step (i).

14. Method according to claim 12 or 13, wherein the efficacy of treatment of the parkinsonian condition is assessed as being positive, if the concentration of 2-hydroxypyridine has decreased compared to the control sample of the same subject or has maintained the same compared to the control sample of the same subject.

15. Method according to claim 12 or 13, wherein the efficacy of treatment of the parkinsonian condition is assessed as being negative, if the concentration of 2-hydroxypyridine has increased compared to the control sample of the same subject, preferably wherein the concentration of 2-hydroxypyridine has significantly increased compared to the control sample of the same subject.

16. Kit-of-parts for determining whether or not a subject has a parkinsonian condition, comprising means for determining the concentration of 2-hydroxypyridine in a sample obtained from the subject and a test system that is capable of determining whether or not said subject has a parkinsonian condition, based on the result of the determined concentration of 2-hydroxypyridine of said subject compared to a control.
